(19) United States
(12) Patent Application Publication
(10) Pub. No.: US 2025/0195864 A1
(43) Pub. Date: Jun. 19, 2025

(54) HELICAL PERICARDIAL ANCHOR SYSTEM

(71) Applicant: PercAssist, Inc., Santa Clara, CA (US)

(72) Inventors: Steven K. KELE, San Jose, CA (US); Hilman H. CHUNG, Mountain View, CA (US); Albert K. CHIN, Palo Alto, CA (US); Sage MORELAND, San Jose, CA (US)

(73) Assignee: PercAssist, Inc., Santa Clara, CA (US)

(21) Appl. No.: 18/952,869

(22) Filed: Nov. 19, 2024

Related U.S. Application Data
(63) Continuation of application No. PCT/US23/67920, filed on Jun. 5, 2023.
(60) Provisional application No. 63/350,716, filed on Jun. 9, 2022.

(51) Int. Cl. A61M 60/187; A61M 60/295; A61M 60/497; A61M 60/515

(52) U.S. Cl. CPC A61M 60/187; A61M 60/295; A61M 60/497; A61M 60/515

(57) ABSTRACT

A pericardial anchor for positioning a cardiac assist system over a patient's heart beneath the patient's sternum and ribs includes a tubular shaft, a pericardial anchor, and a removable handle. The tubular shaft has a guidewire lumen therethrough. The pericardial anchor is positioned at a distal end of the tubular shaft, and the removable handle is positioned at a proximal end of the tubular shaft. The pericardial anchor is typically a helical anchor that can be implanted to anchor in the patient's pericardium by rotation of the handle. The tubular shaft receives and anchors a cardiac assist system over the patient's heart beneath the patient's sternum and ribs after the handle is removed.

### CROSS-REFERENCE

This application is a continuation of PCT Application No. PCT/US23/67920, filed Jun. 5, 2023, which claims priority to U.S. Patent Application No. 63/350,716, filed Jun. 9, 2022, which is incorporated herein by reference.

The subject matter of this patent application is related to the that in the following: U.S. patent application Ser. No. 17/411,928, filed Aug. 25, 2021; PCT Application No. PCT/US2020/019974, filed Feb. 26, 2020, PCT Application No. PCT/US2022/0751, filed Aug. 17, 2022; and U.S. Provisional Patent Application No. 63/407,100, filed Sep. 15, 2022, which are incorporated herein by reference.

### FIELD OF THE PRESENT DISCLOSURE

This disclosure generally relates to devices and methods for placing a helical, spiral, or other anchor into the pericardial membrane to fixate an intrapericardial ventricular assist device post implantation of the anchor. Clockwise rotation of the tip of a rigid spiral placed against the pericardial membrane can cause limited advancement of slightly over one revolution of coil through the pericardium. Limitation of anchor advancement can be critical to the avoidance of injury to the lung during and after its placement.

### BACKGROUND

A balloon cannula may be inserted inside the pericardial sac and positioned anterior to the left ventricle of the heart. Inflation of the balloon during cardiac systole and deflation of the balloon during cardiac diastole may be conducted to increase cardiac output in patients with congestive heart failure. The ventricular assist balloon cannula may be inserted through the pericardium at the inferior aspect of the heart near the apex, via a subxiphoid incision or needle puncture. The distal end of the balloon cannula may be advanced to the left lateral aspect of the heart immediately inferior to the left atrial appendage, resulting in positioning of the balloon anterior to the left ventricle. A fluid tight reservoir may be attached to the proximal end of the balloon cannula, and the reservoir may be implanted subcutaneously in the subxiphoid region. Intrapericardial balloon inflation can be performed via a battery-operated air pump residing outside the body of the patient. A large bore needle may pierce through the patient's skin and the elastomeric sealing face of the subcutaneous reservoir, transmitting flow from the air supply line in the external unit to the intrapericardial balloon cannula.

Upon cyclical inflation and deflation of the balloon, it may be observed that the balloon cannula can migrate out of position over the left ventricle, and it can skew towards the right side of the heart. This may lead to a loss of left ventricular compression and ineffective left ventricular assistance. Therefore, it may be desirable to provide an anchoring system for the tip of the balloon cannula. It is additionally desirable to provide an anchoring system for the ventricular assist balloon cannula that can allow exchange of the balloon cannula while preserving the favorable position established by the original anchor system. The balloon can have a finite life span; for example, one year. Provision of an anchor system that allows balloon cannula exchange may simplify and shorten subsequent balloon replacement procedures.

A trans-pericardial anchor catheter to stabilize the distal end of an intrapericardial balloon cannula has been previously described, comprising of a small diameter non-collapsible catheter body with a short distal section comprised of a braided sheath formed of multiple polymer strands. A rounded tip may be attached to the distal end of the braided sheath, and a stainless-steel wire may be attached to the tip extends the length of the braided sheath and the catheter body. A length of stainless-steel tubing may be bonded to the proximal portion of the catheter body, and the stainless-steel tubing can extend approximately one centimeter proximal to the proximal end of the catheter. The steel wire inside the catheter may be a slip fit with the inner diameter of the stainless-steel tube, and it may protrude several centimeters proximal to the proximal end of the stainless-steel tube. Traction on the stainless-steel wire while the catheter is held stationary can cause the braided sheath to form an expanded disc. The braided sheath may be maintained in its expanded configuration by crimping the stainless-steel tube onto the inner stainless-steel wire.

The previous pericardial anchor catheter may function well as a balloon cannula stabilizing device. However, its placement technique can be hazardous to the patient, as the entire length of the braided sheath typically must exit out of the pericardial sac prior to expansion of the braided sheath to form the anchoring disc. The patient's lung lies very close to the pericardial sac, separated from the pericardium by a few millimeters of extra-pericardial fat and a pleural membrane that is normally less than half of a millimeter thick. The braided sheath length may be greater than 10 mm; therefore, anchor placement may easily cause perforation of the surface of the lung.

### SUMMARY

A pericardial anchor is proposed that involves an elongated, small-bore stainless-steel tube attached to the center of a distal unit comprised of a disc containing a flat distal surface with a spiral rigid coil circumferentially attached to the disc such that a single revolution of the spiral coil extends distal to the flat surface of the disc. The distal portion of the spiral coil may lie in a plane orthogonal to the axis of the tube, and the tip of the spiral may contain an angled undercut, to form a point for pericardial entry. A removeable handle can be provided on the proximal end of the stainless-steel tube. The removeable handle may be a guidewire torquing device, comprises of a short plastic body pin vise, that is clamped onto the stainless-steel tube.

The spiral pericardial anchor system may be inserted into the pericardial sac via an opening formed near the apical aspect of the heart. The opening may be an incision of the pericardium performed via a surgical pericardial window procedure. Alternatively, access into the pericardial sac may be performed percutaneously via needle entry through the skin in the subxiphoid region, advancement of the needle through the pericardium, insertion of a guidewire through the needle, removal of the needle, and advancement of a vascular sheath containing an inner tapered dilator over the guidewire. Upon removal of the tapered dilator and guidewire, the spiral pericardial anchor may be advanced through the vascular sheath into the intrapericardial space. In some situations, the physician may wish to leave the guidewire in position upon removal of the tapered dilator and advance the spiral pericardial anchor system over the guidewire, to guide the anchor system to the desired pericardial location. The pericardial anchor system may employ a stainless-steel tube with an inner lumen to accommodate guidewire placement, rather than a solid stainless-steel wire or rod. Once the pericardial anchor system has been advanced to the desired anchor site, the guidewire may be removed from the lumen of the device, and the spiral tip of the device may be pressed against the pericardium with a constant force of approximately one pound. The spiral may be rotated using the removeable handle multiple revolutions until resistance is met. At this point, one revolution of the spiral anchor has exited the pericardium, and the pericardial membrane has wedged in the apex formed by the flat distal face of the disc and the exposed spiral coil. Resistance felt upon gentle retraction of the anchor system can indicate that proper placement has been achieved.

The elongated stainless-steel tube extending proximally from the spiral coil and the supporting disc may contain a series of radially offset microscopic slots extending one-half of the distal length and the full thickness of the tube. The narrow slots are approximately 0.002″ wide, and they may be formed in the stainless-steel tube using a laser cutter. These slots can impart multi-directional flexibility to the portion of the tube in contact with the heart inside the pericardial sac, to avoid potential myocardial trauma upon long term implantation of the spiral anchor. Adjacent rows of circumferential microscopic slots may be offset with respect to the previous row, imparting axial flexibility while retaining the column strength and torsional stiffness of the tube required for exertion of a one-pound normal force against the pericardium while rotating the spiral to enable its pointed tip to enter the pericardial membrane. Adjacent slots can be spaced a uniform distance for much of the length of the slotted tube. However, the proximal and distal slotted sections may feature adjacent slots that increase linearly in distance as they proceed away from the center of the slotted portion of the tube. Increased distance between slots can provide a strain relief at the point at which the rigid tube becomes flexible, to avoid kinking of the tube at the junction between the solid and slotted sections. The proximal portion of the stainless-steel tube may contain a solid wall, as it often must be rigidly attached to the reservoir of the balloon cannula via a setscrew that compresses and deforms the tube during attachment. A solid proximal section of stainless-steel tube may be required at the reservoir attachment point to ensure proper balloon cannula anchoring, and to avoid any potential of anchor tube fracture over the life of the implant.

An alternate embodiment of the device may employ a stainless-steel tube with an off-round cross section, and a long co-axial slip-fit outer polymer sleeve with a similar cross-sectional profile which is gripped to deploy the spiral anchor. The cross-sectional profile of the stainless-steel tube and the corresponding cross-sectional profile of the outer sleeve keyed to the inner sleeve may be oval, square, hexagonal, or other off-round shape. The increased profile of the outer sleeve can enable it to be used as the grip during spiral anchor placement. A flexible cap may be placed on the proximal end of the stainless-steel tube to stabilize the outer polymer sleeve during spiral anchor placement. Following spiral anchor placement, the proximal end cap may be slipped off the stainless-steel tube, and the outer polymer sleeve removed. This embodiment does not require the use and the removal of the torquer handle component. Torquer grip removal may require the physician to exert a twisting motion of both hands, which may dislodge the spiral anchor from the pericardium. In this embodiment, the flexible cap is pulled axially with the outer sleeve held stationary, thus avoiding any torsional movement that may dislodge the spiral anchor.

The spiral anchor may place one revolution of the spiral through the pericardium. The wire diameter of the spiral is approximately 0.022″ (0.56 mm), and one revolution of the spiral has a depth of approximately 0.056″ (1.4 mm). The mean thickness of the epicardial fat layer in normal individuals without coronary artery disease is 4.4±1.2 mm (Meenakshi K, et al. Epicardial fat thickness: a surrogate marker of coronary artery disease: assessment by echocardiography. 2016; 68:336-341). Therefore, one revolution of spiral at 1.4 mm will not protrude through the epicardial fat layer, and it will stop short of the pleural membrane that surrounds the lung. Hence, lung injury should not occur upon insertion of the spiral anchor into the pericardium.

After placement of the spiral anchor, the ventricular assist balloon cannula can be advanced along the stainless-steel tube component of the anchor into position inside the pericardial sac. An open through-lumen may extend the full length of the balloon, and this through-lumen may accommodate the stainless-steel anchor tube. Following placement of the balloon cannula into position, an implantable reservoir is attached to the proximal end of the cannula. The proximal end of the stainless-steel anchor tube may be inserted into a channel on the side of the reservoir housing, and a setscrew that extends into the channel may be tightened onto the stainless-steel anchor tube to secure the balloon cannula in position throughout the duration of implantation.

Aspects of the present disclosure provide pericardial anchors for positioning a cardiac assist system over a patient's heart beneath the patient's sternum and ribs. An exemplary anchor may comprise a tubular shaft having a guidewire lumen therethrough and a pericardial anchor at a distal end of the tubular shaft. The pericardial anchor may be configured to anchor in the patient's pericardium in response to manipulation of the tubular shaft.

In some embodiments, the pericardial anchor further comprises a removable handle at a proximal end of the tubular shaft. The pericardial anchor may be configured to anchor in the patient's pericardium in response to manipulation of the tubular shaft via the handle. The tubular shaft may be configured to receive and anchor the cardiac assist system over the patient's heart beneath the patient's sternum and ribs after the handle is removed.

In some embodiments, the pericardial anchor further comprises an extension shaft configured to removably couple to the proximal end of the tubular shaft. The extension shaft may have a guidewire lumen. The guidewire lumen of the extension shaft may be co-axial with the guidewire or inner lumen of the tubular shaft. The distal end of the extension shaft may be removably fastened onto the proximal end of the tubular shaft.

In some embodiments, the tubular shaft is a slotted metal tube having controlled flexibility.

In some embodiments, the pericardial anchor is a helical anchor with a flat distal face oriented in a plane orthogonal to an axis of the tubular shaft. The helical anchor may have a sharpened tip configured to penetrate a pericardial membrane with limited penetration into an underlying fat pad.

In some embodiments, the pericardial anchor further comprises a polymer sleeve configured to cover the tubular shaft.

In some embodiments, manipulation of the tubular shaft comprises rotation of the tubular shaft.

Other aspects of the present disclosure also provide methods for positioning a cardiac assist system over a patient's heart beneath the patient's sternum and ribs. An exemplary method may comprise steps of: percutaneously advancing a guidewire to a position over the patient's heart beneath the patient's ribs; advancing a pericardial anchor at a distal end of a tubular shaft over the guidewire to position the pericardial anchor adjacent a preselected location on the patient's pericardium; implanting the pericardial anchor in the pericardium to stabilize the tubular shaft over the patient's heart; and advancing the cardiac assist system over the tubular shaft to locate the cardiac assist system over the patient's heart beneath the patient's sternum and ribs.

In some embodiments, the step of implanting the pericardial anchor in the pericardium comprises rotating the shaft to implant a helical anchor in the pericardium. The helical anchor may have a flat distal face oriented in a plane orthogonal to an axis of the tubular shaft. The helical anchor may have a sharpened tip configured to penetrate a pericardial membrane with limited penetration into an underlying fat pad.

In some embodiments, the cardiac assist system comprises: (i) a pneumatic effector configured to be implanted beneath a patient's pericardial sac and over a myocardial surface overlying the patient's left ventricle; (ii) an implantable port configured to receive a percutaneously introduced cannula, wherein said port is connected to supply a driving gas received from the cannula to the pneumatic effector; (iii) an external drive unit including: (a) a pump assembly; and (b) control circuitry configured to operate the pump to actuate the pneumatic effector in response to the patient's sensed heart rhythm; and (iv) a connecting tube having a pump end attachable to the pump assembly and a cannula end attached to the cannula.

In some embodiments, after the cardiac assist system has been advanced over the tubular shaft to locate the cardiac assist system over the patient's heart beneath the patient's sternum and ribs, the cardiac assist system is locked in position with respect to the tubular shaft to prevent one or more of axial or transverse movement of the cardiac assist system with respect to the heart.

### BRIEF DESCRIPTION OF THE DRAWINGS

FIGS. 1a and 1b illustrate the components comprising the spiral pericardial anchor system and its assembled configuration.

FIGS. 2a-2c depict the geometric configuration of the distal end of the spiral pericardial anchor.

FIGS. 3a and 3b depict the configuration of the partially slotted stainless steel anchor tube.

FIGS. 4a-4c depict an alternate embodiment of a spiral pericardial anchor system.

FIG. 5 illustrates the position of the heart and the left lung, and the anatomical layers of pericardium, extra-pericardial fat, and pleura pertinent to spiral pericardial anchor placement.

FIGS. 6a and 6b are magnified views depicting the process of spiral anchor insertion through the pericardial membrane.

FIG. 7 illustrates the process of vascular sheath placement into position inside the pericardial sac over a previously placed guidewire.

FIG. 8 shows the advancement of the spiral pericardial anchor system through a vascular sheath to guide its placement into the pericardial membrane.

FIG. 9 shows advancement of the ventricular assist balloon cannula over the spiral pericardial anchor embedded in the pericardial membrane.

FIG. 10 shows attachment of the proximal end of the spiral pericardial anchor to the housing of the subcutaneous reservoir attached to the ventricular assist balloon cannula.

### DETAILED DESCRIPTION OF THE DRAWINGS

FIG. 1a depicts an exploded view of the components that form the spiral pericardial anchor system 10 as shown assembled in FIG. 1b. A stainless-steel spiral form 11 with a pointed distal tip may be welded to a stainless-steel bushing 12, and a long stainless-steel tube 13 may be attached to the center of the bushing 12. Spiral structure 11 may be formed of 316 stainless steel, with a wire diameter of approximately 0.022″ and an outer diameter measuring approximately 0.180″. The stainless-steel tube 13 may have an outer diameter of 0.050″ and a wall thickness of 0.005″, such that its inner lumen may accommodate a 0.038″ guidewire. A guidewire torque device 14 may be fastened onto the proximal end of the stainless-steel tube 13 to serve as a handle to facilitate rotation of the spiral pericardial anchor 10 during its placement in the pericardium. The guidewire torque device 14 is shown as a (graspable) block in FIGS. 1a, 1b. Alternatively or in combination, the proximal end of the stainless-steel tube 13 may be fastened onto the distal end of another stainless-steel tube so as to elongate or extend the stainless-steel tube 13. In some cases, the additional stainless-steel tube may be used as the guidewire torque device 14. In some cases, a separate guidewire torque device 14 may be fastened on to the proximal end of the additional stainless-steel tube. The additional stainless-steel tube may have an inner guidewire lumen configured to be coaxial with the inner lumen of the stainless-steel tube 13 as well.

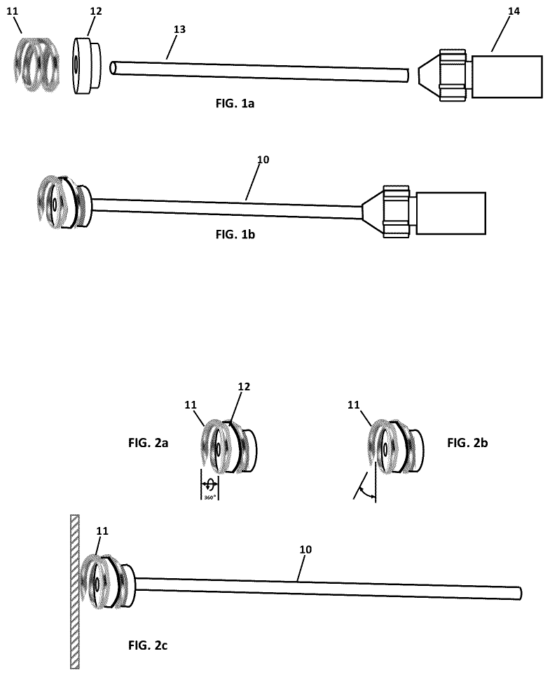

FIG. 2a illustrates the configuration of the spiral 11 as it is attached to the bushing 12. A single revolution of spiral 11 can extend distal to the flat distal face of bushing 12. FIG. 2b shows the configuration of the pointed distal tip of spiral 11, formed by grinding an angle on the inner aspect of the wire tip. An angled undercut of the wire may be used to form the distal tip, such that the distal portion of spiral 11 lies in a flat plane orthogonal to the axis of the spiral anchor system 10, as illustrated in FIG. 2c.

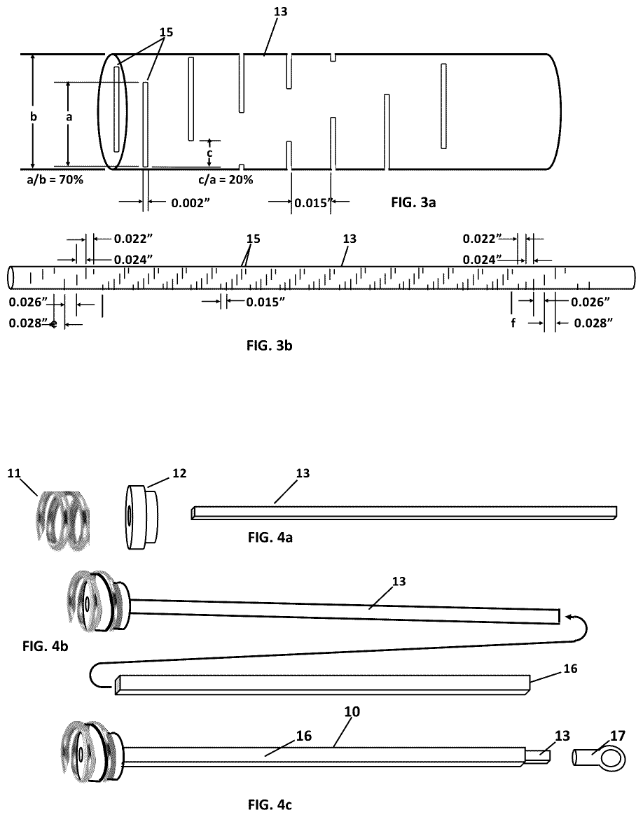

FIG. 3 depicts the configuration of the stainless-steel tube 13, with microscopic slots 15 formed on opposing sides of its wall. The slots 15 may be invisible to the naked eye, at 0.002″ in width, with a length that extends 70% of the 0.050″ diameter of the stainless-steel tube 13, equal to 0.035″. A distance of 0.015″ can separate adjacent axial slots 15. Adjacent slots 15 are radially offset from the previous set of slots 15 by a distance equal to 20% of the length of the slot 15, or 0.035″. The slots 15 may extend one half of the distal length of stainless-steel tube 13. They may impart flexibility to the portion of the stainless-steel tube 13 that lies inside the pericardial sac in contact with the heart. This flexibility may be essential in avoiding trauma to the heart during insertion of the spiral pericardial anchor and upon long term implantation. Excessive rigidity of the stainless-steel tube 13 may cause laceration or perforation of the heart during spiral anchor insertion, as well as potential myocardial laceration during long term implantation. The radially offset series of slots 15 can provide flexibility of the distal stainless-steel tube 13 in all directions without sacrificing the column strength or torsional strength required as the pericardial anchor exerts approximately one pound of force against the pericardium followed by its rotation to achieve pericardial entry and proper anchoring.

FIG. 4a depicts the inner components of an alternate embodiment of a spiral pericardial anchor system 10, comprising of a spiral 11, a bushing 12, and a stainless-steel tube 13. In this embodiment, the stainless-steel tube 13 contains an off-round cross-sectional profile such as a square. FIG. 4b shows that a telescoping, slip-fit polymer sleeve 16 fits over off-round stainless-steel tube 13. The polymer sleeve 16 may contain an inner lumen that matches the outer profile of the off-round stainless-steel tube 13, and it may be slightly shorter in length than stainless steel tube 13. FIG. 4c shows that a flexible end cap 17 may be placed on the exposed proximal end of stainless-steel tube 13 to stabilize polymer sleeve 16 as it is grasped and rotated to insert the anchor system 10 in the pericardium. The proximal portion of polymer sleeve 16 may function as the handle for spiral anchor 11 insertion. Following anchor 11 insertion, the polymer sleeve 16 can be held stationary as the end cap 17 is removed from the stainless-steel tube 13. An advantage of this embodiment may be that it does not require a twisting motion for removal of an attached torque device 14 as in the embodiment shown in FIG. 1b. Two-handed twisting required for removal of torque device 14 may dislodge the spiral 11 from the pericardium. Removal of end cap 17 can involve an axial motion that is less likely to dislodge a fixated spiral 11.

FIG. 5 depicts the heart 18 and left lung 22 in the thoracic cavity. The heart 18 is enclosed by the pericardial sac 19. A pericardial fat pad 20 lies outside of the pericardium 19, and a fibrous pleural membrane 21 encloses the lung 22. The pleura 21 is in contact with the pericardial fat pad 20.

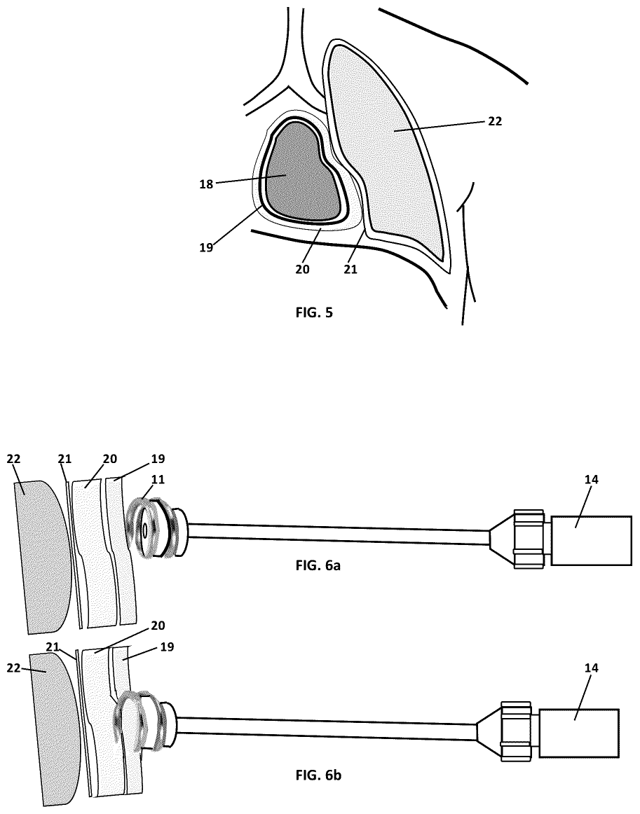

FIG. 6a shows the positioning of the spiral 11 in preparation for insertion into pericardial membrane 19. The extra-pericardial fat pad 20, pleura 21 and lung 22 lie outside of pericardial membrane 19. The spiral 11 may be advanced to be in contact with the pericardial membrane 19 with a mild amount of force, approximately 1 pound of force, and rotated two to three revolutions using the torque handle 14, until resistance is felt, indicating that one revolution of spiral 11 has entered pericardial membrane 19, and pericardial membrane 19 is abutted against the distal face of bushing 12, as seen in FIG. 6b. Human pericardium has a mean thickness of 1.02 mm (Lee J M. Mechanical properties of human pericardium. Circ Res 1985; 55:475), and the pericardial fat pad is approximately 4.4 mm thick. Therefore, the tip of spiral 11 upon placement does not enter the pleura 21 or the lung 22, avoiding potential for perforation or laceration of the lung 22.

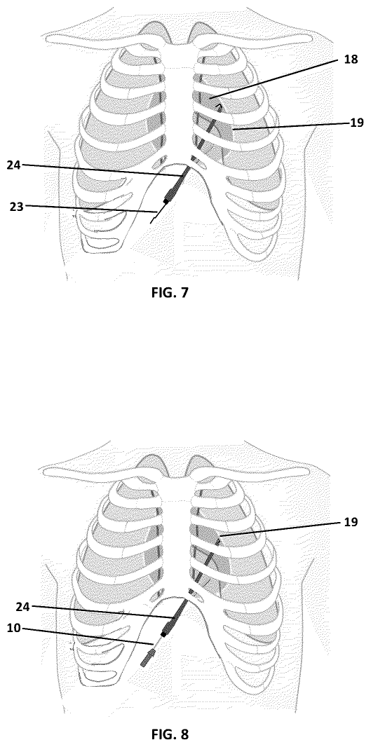

FIG. 7 depicts the positioning of vascular sheath 24 inside the pericardial sac 19 on the left lateral aspect of the heart 18, in preparation for placement of the spiral anchor catheter. Vascular sheath 24 may be advanced over a previously placed guidewire 23 inserted via a needle puncture in the pericardium 19 on the inferior aspect of the heart 18. Positioning of the guidewire 23 and the vascular sheath 24 may be performed under fluoroscopic x-ray guidance.

FIG. 8 shows advancement of the spiral anchor catheter 10 through the vascular sheath 24 positioned at the left lateral border of the pericardial sac 19. Vascular sheath 24 can be held stationary as spiral anchor catheter 10 is rotated to achieve placement through pericardium 19.

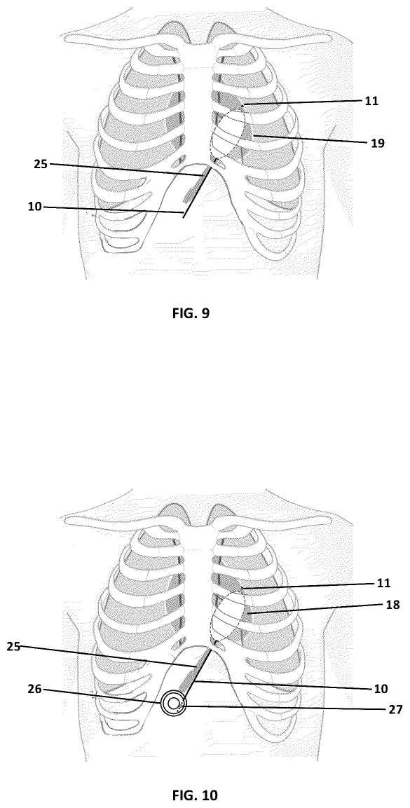

FIG. 9 shows advancement of the ventricular assist balloon cannula 25 along the shaft of spiral anchor catheter 10 after the spiral 11 has been fixated to the pericardial membrane 19. The ventricular assist balloon may be a component of a ventricular assist device as described in related U.S. patent application Ser. No. 17/411,928, filed Aug. 25, 2021, PCT Application No. PCT/US2020/019974, filed Feb. 26, 2020, PCT/US2022/0751, filed Aug. 17, 2022, and U.S. Provisional Patent Application No. 63/407,100, filed Sep. 15, 2022, which are incorporated herein by reference.

FIG. 10 shows that following advancement of ventricular assist balloon cannula 25 over spiral anchor catheter 10, a subcutaneous reservoir 26 may be attached to the proximal end of ventricular assist balloon cannula 25, and the proximal end of spiral anchor catheter 10 may be inserted into a channel in the housing of subcutaneous reservoir 26, where it is locked in position using a setscrew 27. The spiral 11 embedded in the pericardium 19 and the setscrew 27 fixation of the proximal spiral anchor catheter 10 to the reservoir 26 can prevent axial and transverse movement of the ventricular assist balloon cannula 25 with respect to the heart 18.

While preferred embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the invention shall also cover any such alternatives, modifications, variations or equivalents. It is intended that the following claims define the scope of the disclosure and that methods and structures within the scope of these claims and their equivalents be covered thereby.

1. A pericardial anchor for positioning a cardiac assist system over a patient's heart beneath the patient's sternum and ribs, said pericardial anchor comprising:
a tubular shaft having a guidewire lumen therethrough; and
a pericardial anchor at a distal end of the tubular shaft;
wherein the pericardial anchor is configured to anchor in the patient's pericardium in response to manipulation of the tubular shaft.

2. The pericardial anchor of claim 1, further comprising a removable handle at a proximal end of the tubular shaft.

3. The pericardial anchor of claim 2, wherein the pericardial anchor is configured to anchor in the patient's pericardium in response to manipulation of the tubular shaft via the handle.

4. The pericardial anchor of claim 2, wherein the tubular shaft is configured to receive and anchor the cardiac assist system over the patient's heart beneath the patient's sternum and ribs after the handle is removed.

5. The pericardial anchor of claim 1, further comprising an extension shaft configured to removably couple to the proximal end of the tubular shaft.

6. The pericardial anchor of claim 5, wherein the extension shaft has a guidewire lumen.

7. The pericardial anchor of claim 1, wherein the tubular shaft is a slotted metal tube having controlled flexibility.

8. The pericardial anchor of claim 1, wherein the pericardial anchor is a helical anchor with a flat distal face oriented in a plane orthogonal to an axis of the tubular shaft, wherein the helical anchor has a sharpened tip configured to penetrate a pericardial membrane with limited penetration into an underlying fat pad.

9. The pericardial anchor of claim 1, further comprising a polymer sleeve configured to cover the tubular shaft.

10. The pericardial anchor of claim 1, wherein the manipulation of the tubular shaft comprises rotation of the tubular shaft.

11. A method for positioning a cardiac assist system over a patient's heart beneath the patient's sternum and ribs, said method comprising:
percutaneously advancing a guidewire to a position over the patient's heart beneath the patient's ribs;
advancing a pericardial anchor at a distal end of a tubular shaft over the guidewire to position the pericardial anchor adjacent a preselected location on the patient's pericardium;
implanting the pericardial anchor in the pericardium to stabilize the tubular shaft over the patient's heart; and
advancing the cardiac assist system over the tubular shaft to locate the cardiac assist system over the patient's heart beneath the patient's sternum and ribs.

12. The method of claim 11, wherein implanting the pericardial anchor in the pericardium comprises rotating the shaft to implant a helical anchor in the pericardium.

13. The method of claim 12, wherein the helical anchor has a flat distal face oriented in a plane orthogonal to an axis of the tubular shaft, wherein the helical anchor has a sharpened tip configured to penetrate a pericardial membrane with limited penetration into an underlying fat pad.

14. The method of claim 11, wherein the cardiac assist system comprises:
a pneumatic effector configured to be implanted beneath a patient's pericardial sac and over a myocardial surface overlying the patient's left ventricle;
an implantable port configured to receive a percutaneously introduced cannula, wherein said port is connected to supply a driving gas received from the cannula to the pneumatic effector; an external drive unit including:
(a) a pump assembly; and
(b) control circuitry configured to operate the pump to actuate the pneumatic effector in response to the patient's sensed heart rhythm; and
a connecting tube having a pump end attachable to the pump assembly and a cannula end attached to the cannula.

15. The method of claim 11, further comprising locking the cardiac assist system in position with respect to the tubular shaft to prevent one or more of axial or transverse movement of the cardiac assist system with respect to the heart after the cardiac assist system has been advanced over the tubular shaft to locate the cardiac assist system over the patient's heart beneath the patient's sternum and ribs.
